(19) United States
(12) Patent Application Publication
(10) Pub. No.: US 2019/0054005 A1
(43) Pub. Date: Feb. 21, 2019

(54) USE OF ALKYL GLYCOSIDE MODIFIED POLYSILOXANES IN PURE OIL-BASED PERSONAL CARE PRODUCTS

(71) Applicant: Wacker Chemie AG, Munich (DE)

(72) Inventors: Tracy WANG, Zhenjiang, Jiangsu (CN); Eric Xu, Shanghai (CN)

(73) Assignee: Wacker Chemie AG, Munich (DE)

(21) Appl. No.: 16/078,274

(22) PCT Filed: Feb. 24, 2017
(86) PCT No.: PCT/EP2017/054384

§ 371 Date: Aug. 21, 2018

(30) Foreign Application Priority Data
Feb. 29, 2016 (CN) 201610107509.5

(51) Int. Cl. A61K 8/893; A61Q 5/12; A61K 8/895; A61K 8/891; A61K 8/892; A61Q 19/00

(52) U.S. Cl. CPC A61K 8/893; A61Q 5/12; A61K 8/895; A61K 8/891; A61K 2800/594; A61Q 19/007; A61K 2800/31; A61K 2800/54; A61K 8/892

(57) ABSTRACT

Pure oil-based personal care products containing less than or equal to 1 wt. % of water, contain alkyl glycoside modified polysiloxanes of Formula (I)

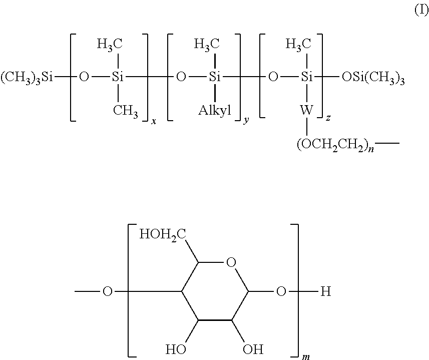

where
x, y and z are between 1 and 10,000,
n and m are between 1 and 20,
alkyl is a linear or branched C₂-C₂₀ hydrocarbon, and
W is a linear or branched C₂-C₂₀ alkylene.

### CROSS REFERENCE TO RELATED APPLICATIONS

This application is the U.S. National Phase of PCT Appln. No. PCT/EP2016/054384 filed Feb. 24, 2017, which claims priority to Chinese Application No. 201610107509.5 filed Feb. 29, 2016, the disclosures of which are incorporated in their entirety by reference herein.

### BACKGROUND OF THE INVENTION

### 1. Field of the Invention

The invention relates to applications of alkyl glycoside modified polysiloxanes in pure oil-based personal care products.

### 2. Description of the Related Art

WO 03/075864 A1 discloses applications of a polyglycerol modified polysiloxane in oily cosmetics, claiming that it is free of stickiness, soft and smooth. The application examples in oily cosmetics include lip sticks, roll-on antiperspirants and oily liquid foundations. In oily liquid foundations therein, however, emulsifiers based on fatty acid esters are needed to help the powder suspend in the systems.

U.S. Pat. No. 6,066,326 discloses a cosmetic composition comprising at least one co-emulsifier and one polydimethylsiloxane containing glucoside groups. In Examples 1-4 disclosed therein, the polydimethylsiloxane containing glucoside groups is used in water-in-oil emulsions.

U.S. Pat. No. 6,881,416B2 discloses a process for the preparation of water-in-oil emulsions used as foundations by mixing WACKER BELSIL SPG 128 VP and WACKER BELSIL RG 100 with powders.

U.S. Pat. No. 5,831,080 describes a process for preparing a glycoside modified polysiloxane.

U.S. Pat. No. 6,919,071 discloses a water-in-oil sunscreen emulsion, where glycoside modified siloxane(s) is/are used as emulsifier(s).

WO2015091378A discloses a novel alkyl glycoside polysiloxane elastomer gel, whose crosslinked molecular chains contain silicone resin segments.

### SUMMARY OF THE INVENTION

It has now been surprisingly discovered that alkyl glycoside modified polysiloxanes can be employed in pure oil-based personal care products to significantly reduce the stickiness of the pure oil-based products, in pure oil-based hair conditioner compositions to significantly improve the smoothness of hair, and in pure oil-based skin care compositions containing powders to significantly enhance the stability of compositions having a high powder content and to impart them with an excellent powder suspensibility.

### DETAILED DESCRIPTION OF THE PREFERRED EMBODIMENTS

The pure oil-based personal care products of the invention include pure oil-based hair conditioner compositions (including leave-on hair conditioner compositions), and pure oil-based skin care compositions (including facial skin care compositions, particularly those having a high powder content).

“Pure oil-based” in this invention means that the pure oil-based products comprise less than or equal to 1 wt % of water, preferably less than or equal to 0.1 wt % of water. These may be compositions mainly containing oil ingredients and water content less than or equal to 1 wt %, preferably less than or equal to 0.1 wt % of water.

The leave-on hair conditioner compositions in the invention refer to hair conditioner compositions that, after applying to hair, have a retention time greater than or equal to 1 hour. Preferably, “Leave-on” means that the hair is not rinsed until 8 hours after applying the hair conditioner.

The volatile hydrophobic solvents in the invention refer to hydrophobic solvents whose evaporation rate is greater than or equal to 0.1 wt %/min tested at 23° C. according to DIN53249: 2007-05 and which are typically suitable for personal care products, including cyclopentasiloxane, isododecane and isohexadecane. “Hydrophobic” means that the solubility of the solvent in water is equal or less than 1 wt % at 25° C. at 1013 hPa.

The nonvolatile oils in the invention refer to the oils whose evaporation rate is less than 0.1 wt %/min tested at 23° C. according to DIN53249, including organosilicon oils such as dimethiconols (INCI name), which are α,ω-dihydroxy-terminated polydimethylsiloxanes, preferably with a viscosity of from 0.1 to 2,000 mPas at 25° C. (according to DIN 53019), dimethicones (INCI name), which are dimethyl polysiloxanes, preferably with a viscosity of from 0.1 to 2,000 mPas at 25° C. (according to DIN 53019), and phenyl modified dimethicones (INCI name), which are phenyl modified dimethyl polysiloxanes, preferably with a viscosity of from 0.1 to 2,000 mPas at 25° C. (according to DIN 53019); hydrocarbon oils such as white spirit (a mixture of aliphatic and alicyclic C7 to C12 hydrocarbons), liquid paraffin, micro-crystalline wax, beeswax, C12-15 benzoates, palmitic acid, panthenol, cetanol, myristyl alcohol, isopropyl palmitate, sorbitan stearate, ceteareth-3, glyceryl laurates and ethylhexyl methoxycinnamate; vegetable oils such as sunflower oil, castor oil, jojoba oil, Shea butter, avocado and grape seed oils; and animal fats such as lanolin and mink oils.

INCI names are described in the “International Cosmetic Ingredient Dictionary & Handbook”, 16^{th} Edition, from the Personal Care Product Council (ed.).

The powders for personal care products in the invention refer to inorganic and polymeric powders with nano-or micron-level particle size used to adjust colors or block UV in the field of personal care products, including silica, talc, mica, titanium dioxide, color pigment powders (such as iron oxide yellow, iron oxide black, iron oxide red, etc.), kaolin, zinc oxide and polymethacrylate.

The alkyl glycoside modified polysiloxanes in the invention can be prepared by the method as described in CN102492147B. The alkyl glycoside modified polysiloxanes in the invention are not crosslinked polymers and have a linear polysiloxane backbone with part of side groups being substituted by alkyl and glycoside groups.

The invention can be realized as follows:

The invention provides for the use of alkyl glycoside modified polysiloxanes in pure oil-based personal care products, wherein the pure oil-based products comprise less than or equal to 1 wt % of water, preferably less than or equal to 0.1 wt % of water, and the structure of the alkyl glycoside modified polysiloxanes is as shown in Formula (I)

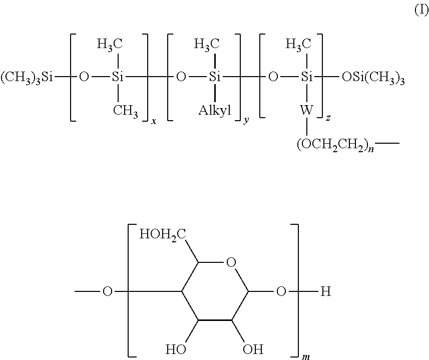

where x, y and z are the same or different and respectively represent positive integers between 1 and 10,000, preferably positive integers between 1 and 5,000;

n and m are the same or different and respectively represent positive integers between 1 and 20, preferably positive integers between 1 and 15;

Alkyl represents a linear or branched C₂-C₂₀ hydrocarbon chain;

with preference given to at least one alkyl group selected from a group of ethyl, propyl, butyl, pentyl, hexyl, heptyl and octyl;

W represents a linear or branched C₂-C₂₀ alkylene selected from a group of ethylene, propylene, butylene, pentylene, hexylene, heptylene and octylene.

According to the use as mentioned above, the alkyl glycoside modified polysiloxanes are preferably used in amounts of from 0.1 to 50 wt % in pure oil-based personal care products.

As in the application as mentioned above, the pure oil-based personal care products are preferably leave-on hair conditioner compositions having a turbidity of less than 80 NTU (Nephelometric Turbidity Unit), preferably less than 30 NTU, tested according to ISO 7027-1:2016 standard, measured preferably by the turbidimeter HACH-2100Q and its user instructions at room temperature.

According to the use as mentioned above, the pure oil-based hair conditioner compositions preferably contain:

(a) 0.1-50 wt % of alkyl glycoside modified polysiloxanes;

(b) 0-85 wt % of volatile hydrophobic solvents;

(c) 0-20 wt % of non-volatile oils;

(d) 0.1-10 wt % of dimethicone/vinyl dimethicone crosspolymer

where the amount of components (b) and (c) cannot be zero at the same time and the percentage in each case is based on the total weight of the pure oil-based compositions.

Dimethicone/vinyl dimethicone crosspolymer (INCI name) is a crosslinked dimethyl siloxane polymer, preferably a silicone elastomer, formed by the reaction between a dimethyl polysiloxane having Si-bonded hydrogen and a dimethyl polysiloxane having Si-bonded vinyl groups, for example by the reaction of hydrogen dimethicone (INCI name; dimethyl polysiloxane having Si-bonded hydrogen) or bis-hydrogen dimethicone (INCI name; α,ω-dihydrogen dimethyl polysiloxane), and vinyl dimethicone (INCI name, dimethyl polysiloxane having Si-bonded vinyl groups) or bis-vinyldimethicone (INCI name, α,ω-divinyl dimethyl polysiloxane).

According to the use as mentioned above, the pure oil-based hair conditioner compositions preferably contain:

(a) 0.1-30 wt % of alkyl glycoside modified polysiloxanes;

(b) 20-85 wt % of volatile hydrophobic solvents;

(c) 5-35 wt % of non-volatile oils containing 0.5-35 wt % of dimethiconols;

where the percentage in each case is based on the total weight of the pure oil-based composition.

According to the use as mentioned above, the pure oil-based hair conditioner compositions preferably contain:

(a) 0.1-30 wt % of alkyl glycoside modified polysiloxanes;

(b) 30-85 wt % of volatile hydrophobic solvents;

(c) 5-30 wt % of non-volatile oils containing 0.5-30 wt % of dimethiconols;

(d) 0.5-10 wt % of dimethicone/vinyl dimethicone crosspolymer

where the percentage in each case is based on the total weight of the pure oil-based composition.

According to the use as mentioned above, the pure oil-based personal care products are preferably skin care compositions, more preferably facial skin care compositions.

According to the use as mentioned above, the pure oil-based skin care compositions preferably contain:

(a) 0.1-10 wt % of alkyl glycoside modified polysiloxanes;

(b) 10-90 wt % of volatile hydrophobic solvents;

(c) 5-80 wt % of non-volatile oils containing 0.5-10 wt % of dimethiconols;

(d) 0.1-10 wt % of dimethicone/vinyl dimethicone crosspolymer

where the percentage in each case is based on the total weight of the pure oil-based composition.

According to the use as mentioned above, the pure oil-based skin care compositions preferably contain:

(a) 0.1-10 wt % of alkyl glycoside modified polysiloxanes;

(b) 10-70 wt % of volatile hydrophobic solvents;

(c) 19-80 wt % of non-volatile oils containing 0.5-10 wt % of dimethiconols;

(d) 0-10 wt % of dimethicone/vinyl dimethicone crosspolymer

(e) 0.5-50 wt % of powders that can be used in the skin care compositions;

where the percentage in each case is based on the total weight of the pure oil-based composition.

According to the use as mentioned above, the pure oil-based skin care compositions preferably contain:

(a) 0.1-10 wt % of alkyl glycoside modified polysiloxanes;

(b) 10-70 wt % of volatile hydrophobic solvents;

(c) 19-75 wt % of non-volatile oils containing 0.5-10 wt % of dimethiconols;

(d) 0.1-10 wt % of dimethicone/vinyl dimethicone crosspolymer

(e) 10-50 wt % of powders that can be used in the skin care compositions;

where the percentage in each case is based on the total weight of the pure oil-based composition.

The components (a), optionally (b), optionally (c) and optionally (d) and optionally (e) are used in each case in such amounts that they add up to 100 wt %.

### DESCRIPTION OF THE PREFERRED EMBODIMENTS

The information on the products herein is as follows:

BELSIL® GB 1020 by Wacker Chemie AG, comprising 12 wt % of dimethiconols and 88 wt % of dimethicones, wherein the viscosity of dimethicones is 5 mPa·s (at 25° C. according to DIN 53019).

BELSIL® EG 2 by Wacker Chemie AG, comprising 14 wt % of dimethicone/vinyl dimethicone crosspolymers and 86 wt % of cyclopentasiloxane.

BELSIL® SPG 128 VP by Wacker Chemie AG, comprising 20 wt % of caprylyl dimethicone ethoxy glucoside and 80 wt % of cyclopentasiloxane.

BELSIL® WO 5000 by Wacker Chemie AG, comprising 30 wt % of caprylyl dimethicone ethoxy glucoside and 70 wt % of dimethicones, wherein the viscosity of dimethicone is 5 mPa·s (at 25° C. according to DIN 53019).

BELSIL® DM 5 by Wacker Chemie AG, comprising 100 wt % of dimethicones having a viscosity of 5 mPa·s (at 25° C. according to DIN 53019).

BELSIL® CM 040 CN by Wacker Chemie AG, comprising 100 wt % of cyclopentasiloxane.

Wacker Chemie AG's alkyl glycoside polysiloxane elastomer gel samples, whose polysiloxane molecular chains contain silicone resin segments, and which are prepared in reference to Sample 14 in Table 5 of WO2015091378A and contain alkyl glycoside polysiloxane elastomers.

KF-6104 by ShinEtsu, comprising polyglyceryl-3 polydimethylsiloxyethyl dimethicone.

BELSIL® PDM 20 by Wacker Chemie AG, comprising 100 wt % of trimethlysiloxyphenyl dimethicone

Landpowder Si BB88231 by Shanghai Kingland Fine Chemistry's Co., Ltd., a mixture of titanium dioxide and color pigment powders.

Pure Oil-Based Hair Conditioner Compositions

The following ingredients were mixed and stirred at a medium rotating speed of 200-300 rpm at room temperature to obtain Examples 1-3 (abbreviation Ex. 1-3) and Comparative Examples 1-3 (abbreviation C. Ex. 1-3).

TABLE 1

| | | Pure oil-based hair | | | | | |
|---|---|---|---|---|---|---|---|
| | | conditioner compositions | | | | | |
| | | | C. | | C. | C. | |
| | Material | Ex. 1 | Ex. 1 | Ex. 2 | Ex. 2 | Ex. 3 | Ex. 3 |
| INCI Names | from | wt % | wt % | wt % | wt % | wt % | wt % |
| Dimethicone | BELSIL ® | 70 | 70 | 15.0 | 15.0 | 23.0 | 15.0 |
| | DM 5 | | | | | | |
| Polyglyceryl-3 | KF-6104 | / | 30.0 | / | / | / | / |
| polydimethyl- | | | | | | | |
| siloxyethyl | | | | | | | |
| dimethicone | | | | | | | |
| Alkyl glycoside | Alkyl | / | / | / | / | 2.0 | / |
| polysiloxane | glycoside | | | | | | |
| elastomer | polysiloxane | | | | | | |
| | elastomer | | | | | | |
| | gel sample | | | | | | |
| Dimethiconol | BELSIL ® | / | / | 1.2 | / | / | 1.2 |
| | GB 1020 | | | | | | |
| Cyclopenta- | BELSIL ® | / | / | 81.8 | 83.0 | 75.0 | 79.0 |
| siloxane | CM 040 CN | | | | | | |
| dimethicone/ | BELSIL ® | / | / | / | / | / | 2.8 |
| vinyl | EG 2 | | | | | | |
| dimethicone | | | | | | | |
| crosspolymer | | | | | | | |
| Caprylyl | BELSIL ® | / | / | 2.0 | 2.0 | / | 2.0 |
| dimethicone | SPG 128 VP | | | | | | |
| ethoxy | | | | | | | |
| glucoside | | | | | | | |
| Caprylyl | BELSIL ® | 30.0 | / | / | / | / | / |
| dimethicone | WO 5000 | | | | | | |
| ethoxy | | | | | | | |
| glucoside | | | | | | | |

Tested according to ISO7027 standard, each of the 3 compositions prepared in Examples 1-3 herein had a turbidity of less than 3ONTU.

Naturally straight Chinese hair, never dyed or permed, was used for experimental purposes in the invention, and each hair tress measured 26 cm long and weighed 12 g. 1.4 ml of Pantene Pure Nourish (product number 98573610) Shampoo commercially available in 2015 was used to wash each straight hair tress, which was then air dried.

In Examples 1-3 and Comparative Examples 1-3, 0.7 ml of individual care products was evenly applied to the corresponding hair tress which has been cleaned and dried as above. The period of 10 seconds starting from applying the care products on the hair tress was defined as the early stage of application. The period following 60 seconds after the care products were applied on the hair tress was defined as the later stage of application.

A rating system is as follows:

| | Performance rating | | | | |
|---|---|---|---|---|---|
| | 1 | 2 | 3 | 4 | 5 |
| Hair | Not smooth at all; | Not very | Average | Smooth in reference | Very |
| smoothness | typical effect with | smooth | | to the effect from using | smooth |
| in the | no care product | | | the same amount of | |
| early stage | being used; significant | | | L'ORÉAL Extraordinary | |
| of application | resistance when | | | Oil 2K060 | |
| | touching hair tresses | | | | |
| | following the growth | | | | |
| | direction of hair | | | | |
| | cuticles | | | | |
| Hair | Very sticky in | Sticky | Average | Not very sticky in | Not |
| stickiness | reference to the | | | reference to the effect from | sticky |
| in the | effect from using | | | using the same amount of | |
| later stage | the same amount of | | | L'ORÉAL Extraordinary | |
| of application | lanolin wax | | | Oil 2K060 | |
| Hair | Not fluffy in | Slightly | Average | Fluffy in reference | Very |
| fluffiness | reference to the | fluffy | | to the effect from using | fluffy |
| in the | effect from using | | | the same amount of | |
| later stage | the same amount of | | | L'ORÉAL Extraordinary | |
| of application | lanolin wax | | | Oil 2K060 | |

Property values of the pure oil-based hair conditioner compositions of the examples and comparative examples are listed in Table 2.

TABLE 2

| | | C. | | C. | C. | |
|---|---|---|---|---|---|---|
| | Ex. 1 | Ex. 1 | Ex. 2 | Ex. 2 | Ex. 3 | Ex. 3 |
| Hair | 5 | 3 | 5 | 4 | 3 | 5 |
| smoothness in | | | | | | |
| the early | | | | | | |
| stage of | | | | | | |
| application | | | | | | |
| Hair | 5 | 3 | 5 | 5 | 5 | 5 |
| stickiness in | | | | | | |
| the later | | | | | | |
| stage of | | | | | | |
| application | | | | | | |
| Hair | 1 | 1 | 1 | 1 | 2 | 4 |
| fluffiness in | | | | | | |
| the later | | | | | | |
| stage of | | | | | | |
| application | | | | | | |

The hair tress was not smooth after treated with the product prepared in Comparative Example 3. The reason for this is that the alkyl glycoside polysiloxane elastomer has nonlinear polysiloxane molecular chains, and the silicone segments cannot contribute to smoothness effect. In addition, the product obtained in Example 3 by the addition of dimethicone/vinyl dimethicone crosspolymer significantly increased the fluffiness of hair.

### Example 4

| | Trade names of ingredients and | | |
|---|---|---|---|
| Phase | substances contained | Suppliers | wt % |
| A | BELSIL ® SPG 128 VP containing | Wacker Chemie | 10.0 |
| | 20 wt % of caprylyl dimethicone | | |
| | ethoxy glucoside | | |
| | BELSIL ® GB 1020 containing | Wacker Chemie | 30.0 |
| | 12 wt % of dimethiconol | | |
| | BELSIL ® PDM 20 containing | Wacker Chemie | 5.0 |
| | 100 wt % of trimethlysiloxyphenyl | | |
| | dimethicone | | |
| | IDD containing 100 wt % of | DowPol | 50.0 |
| | isododecane | | |
| | IPP containing 100 wt % of | BASF | 5.0 |
| | ethylhexyl palmitate | | |

In Example 4, the smoothness effect of the product was significantly improved by the addition of 3.6 wt % of dimethiconol, based on the total amount of composition.

Pure Oil-Based Skin/Face Care Compositions

To prepare the skin massage oils of Example 5 and Comparative Example 5, oil phase ingredients were mixed one by one and stirred at a medium speed of 200-300 rpm at room temperature. To prepare the oily liquid foundations of Examples 6 and 7 and Comparative Examples 6 and 7, oil phase ingredients were mixed one by one and stirred well at a medium speed at room temperature. Afterwards, the powder was added to and stirred with the mixture of oils at a medium speed of 200-300 rpm for 2 minutes and then homogenized at 10,000 rpm for 3 minutes.

TABLE 3

| | | Pure oil-based | | | | | |
|---|---|---|---|---|---|---|---|
| | | skin care compositions | | | | | |
| | | | C. | | C. | C. | |
| | Material | Ex. 5 | Ex. 5 | Ex. 6 | Ex. 6 | Ex. 7 | Ex. 7 |
| INCI Names | from | wt % | wt % | wt % | wt % | wt % | wt % |
| Dimethicone | BELSIL ® | 10.0 | 10.0 | 10.0 | 10.0 | 18.0 | 10.0 |
| | DM 5 | | | | | | |
| Polyglyceryl-3 | KF-6104 | / | 0.2 | / | 2.0 | / | / |
| polydimethyl- | | | | | | | |
| siloxyethyl | | | | | | | |
| dimethicone | | | | | | | |
| Alkyl glycoside | Alkyl | / | / | / | / | 2.0 | / |
| polysiloxane | glycoside | | | | | | |
| elastomer | polysiloxane | | | | | | |
| | elastomer | | | | | | |
| | gel sample | | | | | | |
| Dimethiconol | BELSIL ® | / | / | 0.5 | 0.5 | 0.5 | 0.5 |
| | GB 1020 | | | | | | |
| Cyclopenta- | BELSIL ® | 89.8 | 89.8 | 72.5 | 72.5 | 64.5 | 69.7 |
| siloxane | CM 040 CN | | | | | | |
| 0-10 wt % of | BELSIL ® | / | / | / | / | / | 2.8 |
| dimethicone/ | EG 2 | | | | | | |
| vinyl | | | | | | | |
| dimethicone | | | | | | | |
| crosspolymer | | | | | | | |
| Caprylyl | BELSIL ® | 0.2 | / | 2.0 | / | / | 2.0 |
| dimethicone | SPG 128 VP | | | | | | |
| ethoxy | | | | | | | |
| glucoside | | | | | | | |
| Titanium | SiBB2231 | / | / | 15.0 | 15.0 | 15.0 | 15.0 |
| dioxide | | | | | | | |
| powder | | | | | | | |

The skin and face care compositions prepared in Examples 5-7 and Comparative Examples 5-7 herein were applied to the inside of the arm with gentle massage in circular motions using the middle finger until absorbed completely, and evaluated for their stickiness or smoothness 5 minutes later.

The stickiness (hand feel) was evaluated in reference to the following:
The stickiness of lanolin wax was given 10 points (very sticky);
Jergens Aloe and Lanolin (commercial product) oil-in-water lotion was given 5 points (moderately sticky); The stickiness of baby oil (pure mineral oil) was given 0 point (not significantly sticky).

The smoothness was evaluated in reference to the following:
Talcum powder as a reference was given 10 points (significantly smooth);
Jergens Aloe and Lanolin (commercial product) was given 5 points (moderately smooth);
Water as a reference was given 0 point (not significantly smooth);

The compositions were evaluated for their storage stabilities, especially powder suspensibility, in reference to the following:
Fully suspended powder having no layering or phase separation after letting stand for 4 hours at room temperature was given 10 points;
Moderately suspended powder having slight layering without any clear interface after letting standing for 4 hours at room temperature was given 5 points;
Non-suspended powder having a clear layered interface within 1 hour after letting stand at room temperature was given 0 point.

Property values of the pure oil-based face-care compositions of examples and comparative examples are listed in Table 4. The experiments showed that polyglyceryl-3 polydimethylsiloxyethyl dimethicone and alkyl glycoside polysiloxane elastomer did not show the ability to help with powder suspensibility in Comparative Example 6 and 7. In Examples 6 and 7, 2 wt % of caprylyl dimethicone ethoxy glucoside was used to obtain a homogeneous and stable pure oil-based liquid foundation having no layering or phase separation and a power content up to 15 wt %.

TABLE 4

| | | C. | | C. | C. | |
|---|---|---|---|---|---|---|
| | Ex. 5 | Ex. 5 | Ex. 6 | Ex. 6 | Ex. 7 | Ex. 7 |
| Stickiness | 6 | 7 | 5 | 6 | 5 | 5 |
| Smoothness | 7 | 6 | 7 | 5 | 7 | 8 |
| Powder | / | / | 10 | 0 | 0 | 10 |
| suspensibility | | | | | | |

### EXAMPLE 8

### Massage Oil for Skin Care

| | Trade names of ingredients and | | |
|---|---|---|---|
| Phase | substances contained | suppliers | wt % |
| A | IDD containing 100 wt % of isodode- | DowPol | 50.0 |
| | cane | | |
| | BELSIL ® GB 1020 containing 12 | Wacker Chemie | 25.0 |
| | wt % of dimethiconol | | |
| | BELSIL ® SPG 128 VP containing 20 | Wacker Chemie | 10.0 |
| | wt % of caprylyl dimethicone ethoxy | | |
| | glucoside | | |
| | Olive oil | Lipo Chemicals | 5.00 |
| | Jojoba oil | Blue Star | 5.00 |
| | Macadamia oil | Lipo Chemicals | 5.00 |

The product prepared in Example 8 contained 3 wt % of dimethiconol and scored 8 points for its smoothness, which was significantly improved.

1.-10. (canceled)

11. Pure oil-based personal care products comprising less than or equal to 1 wt. % of water, contain alkyl glycoside modified polysiloxanes of the 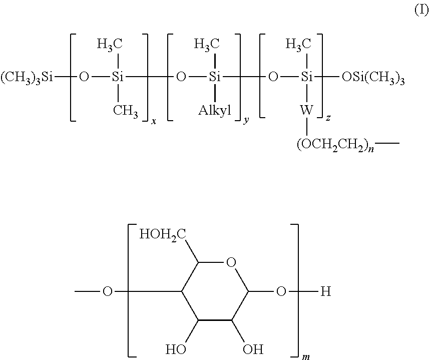
Formula (I)
where
x, y and z are the same or different and are positive integers between 1 and 10,000,
n and m are the same or different and are positive integers between 1 and 20,
alkyl represents a linear or branched C₂-C₂₀ hydrocarbon group;
W are linear or branched C₂-C₂₀ alkylenes selected from the group consisting of ethylene, propylene, butylene, pentylene, hexylene, heptylene and octylene,
wherein the pure oil-based personal care products are pure oil-based hair conditioner compositions containing:
(a) 0.1-50 wt % of alkyl glycoside modified polysiloxanes;
(b) 0-85 wt % of volatile hydrophobic solvents;
(c) 0-20 wt % of non-volatile oils;
(d) 0.1-10 wt % of dimethicone/vinyl dimethicone crosspolymer
where the amount of components (b) and (c) cannot be zero at the same time and the percentages in each case are based on the total weight of the pure oil-based compositions.

12. The personal care product of claim 11, wherein x, y, and z are between 1 and 5000.

13. The personal care product of claim 11, wherein n and m are between 1 and 15.

14. The personal care product claim 12, wherein n and m are between 1 and 15.

15. The personal care product of claim 11, wherein the pure oil-based personal care products are leave-on hair conditioner compositions having a turbidity of less than 80 NTU (Nephelometric Turbidity Unit) tested according to ISO 7027-1:2016 standard.

16. The personal care product of claim 11, wherein the pure oil-based personal care products are leave-on hair conditioner compositions having a turbidity of less than 30 NTU (Nephelometric Turbidity Unit) tested according to ISO 7027-1:2016 standard.

17. The personal care product of claim 11, wherein the pure oil-based hair conditioner compositions comprise:
(a) 0.1-30 wt % of alkyl glycoside modified polysiloxanes;
(b) 30-85 wt % of volatile hydrophobic solvents;
(c) 5-30 wt % of non-volatile oils containing 0.5-30 wt % of dimethiconols;
(d) 0.5-10 wt % of dimethicone/vinyl dimethicone crosspolymer;
where the percentage in each case is based on the total weight of the pure oil-based composition.

18. The personal care product of claim 11, containing less than or equal to 0.1 wt. % water.
